(19) United States
(12) Patent Application Publication
(10) Pub. No.: US 2008/0249548 A1
(43) Pub. Date: Oct. 9, 2008

(54) Corneal graft preparation

(76) Inventor: Philip Douglas Weston, Ledyard, CT (US)

Correspondence Address: IRA S. DORMAN, SUITE 200, 330 ROBERTS STREET, EAST HARTFORD, CT, 06108, US

(21) Appl. No.: 12/080,194

(22) Filed: Apr. 1, 2008

Related U.S. Application Data
(60) Provisional application No. 60/922,029, filed on Apr. 5, 2007.

(30) Foreign Application Priority Data
Apr. 4, 2007 (GB) GB0706608.7
Mar. 10, 2008 (GB) PCT/GB2008/050166

(51) Int. Cl. A61F 9/007

(52) U.S. Cl. 606/166

(57) ABSTRACT

There is disclosed a method of separating a posterior endothelial layer of a donor cornea having an epithelium, layers of stroma, a Descemet's membrane and an endothelium, after the donor cornea has been harvested from a cadaver. The method comprises the steps of mounting the donor cornea on an artificial anterior chamber provided with a datum point and means for receiving a microkeratome and using the microkeratome to separate the posterior endothelial layer of the donor cornea. The artificial anterior chamber is configured such that the datum point is located at a predetermined height relative to the endothelium when the donor cornea is mounted, and a cutting height of the microkeratome is set to a predetermined height with reference to the datum point. There is also disclosed an artificial anterior chamber and a microkeratome for use with the method.

### CROSS REFERENCE TO RELATED APPLICATIONS

This application claims the benefit of U.S. Provisional Patent Application No. 60/922,029, filed Apr. 5, 2007. This application also claims the benefit of International Application No. PCT/GB2008/050166, filed Mar. 10, 2008. The entire specifications of the above-identified United States and International applications are hereby incorporated hereinto by reference thereto.

Embodiments of the present invention relate to surgical devices adapted for preparing corneal grafts from donor corneal tissue harvested from a cadaver. Embodiments of the invention also relate to methods of preparing such corneal grafts. It is important in the context of the present application to understand that the devices of embodiments of the invention hereinbelow described are adapted to operate on corneal tissue harvested from a cadaver, and are not intended for surgical use in situ on a living patient. Moreover, methods of embodiments of the present invention are not to be understood as surgical techniques performed on the human or animal body, but are instead techniques for obtaining predominantly endothelial grafts from a donor cornea after this has been harvested or separated from the eye of a cadaver.

### BACKGROUND

The human cornea is a multilayered structure, having five principal layers. From the anterior to posterior they are:

Corneal epithelium: a thin epithelial multicellular layer of fast-growing and easily-regenerated cells, kept moist with tears.

Bowman's layer (also erroneously known as the anterior limiting membrane, when in fact it is not a membrane but a condensed layer of collagen): a tough layer that protects the corneal stroma, consisting of irregularly-arranged collagen fibres.

Corneal stroma (also substantia propria): a thick, transparent middle layer, consisting of regularly-arranged collagen fibers along with sparsely populated keratocytes. The corneal stroma consists of approximately 200 layers of type I collagen fibrils.

Descemet's membrane (also posterior limiting bane): a thin a cellular layer that serves as the modified basement-membrane of the corneal endothelium.

Corneal endothelium: a simple squamous or low cuboidal monolayer of mitochondria-rich cells responsible for regulating fluid and solute transport between the aqueous and corneal stromal compartments.

Microkeratomes for cutting layers from corneas are well known. A typical example is disclosed in U.S. Pat. No. 6,022,365, the full contents of which is hereby incorporated into the present application by reference. This microkeratome, in contrast to those of embodiments of the present invention, is intended for application to the eye of a living patient, and is designed to cut a corneal epithelial flap which can then be lifted by a surgeon prior to laser ablation of the corneal stroma in order to correct the refractive index of the cornea as a whole. The flap is then put back into place after surgery so as to promote rapid healing and recovery.

In this type of surgery, often referred to as Laser-Assisted In Situ Keratomileusis (LASIK), it is desired to cut only an epithelial flap from the anterior surface of the cornea. The cutting depth is carefully determined prior to surgery by using an expensive optical or ultrasound rangefinding device so as to determine a cutting depth with reference to the anterior surface of the epithelial surface. This is especially important since the surgery is performed on a living patient.

As previously mentioned, embodiments of the present invention are directed towards devices for cutting corneal grafts from donor corneal tissue. These grafts can then be used to replace diseased corneal tissue in a living patient. In order to prepare a corneal graft, it is known to harvest a donor cornea by any conventional means, and then to process it using an artificial anterior chamber, for example as disclosed in U.S. Pat. No. 6,045,563, the full contents of which is hereby incorporated into the present application by reference. The donor cornea will generally comprise a cornea-scleral disk, the disk comprising a cornea surrounded by a scleral ring. The disk is generally stored in an appropriate conservation liquid prior to use. The disk is then mounted in an artificial anterior chamber, with its epithelial surface uppermost and the scleral ring clamped appropriately, for example by way of a clamping ring and screw thread. A fluid pressure inside the artificial anterior chamber is adjusted so as to stiffen the cornea and present an appropriate degree of curvature, and a microkeratome is mounted on the chamber and used to cut an anterior layer (comprising the epithelium and most of the stroma) from the cornea so as to leave a thin posterior layer of stroma, Descemet's membrane and the endothelium. A central button is then cut from the posterior layer using a donor corneal punch or other suitable device, and the endothelial button can then be used to replace diseased endothelial tissue in a living patient. This is done by making a small incision in the eye of the patient with a keratome, cutting out the diseased endothelium and attached Descemet's membrane using a micro-surgical hook and a Descemet's stripper, and then rolling up the endothelial button before inserting this through the incision and unfurling it carefully so as to adhere to the underside of the patient's cornea.

This surgical technique is generally known as Descemet's Stripping Automated Endothelial Keratoplasty (DSAEK). This term tends to be preferred where a motorised microkeratome is used. Another term is Deep (Stromal) Lamellar Endothelial Keratoplasty (DLEK or DSLEK).

Known prior art microkeratome and artificial anterior chamber combinations are designed and set up so as to cut to a predetermined depth as measured from the surface of the epithelium. In other words, a datum point from which cutting depths are measured is defined as a point relative to the surface of the epithelium. This is because all known microkeratomes used for DSAEK procedures are adapted directly from devices designed for LASIK anterior surgical procedures.

However, the thickness from the epithelium down to the endothelium varies from person to person, and also varies with a person's age and even varies with time during the day. This makes accurate cutting very difficult, since the cutting depth is not known ab initio with accuracy. It is possible to measure the required cutting depth by using optical (e.g. laser) measuring devices and calibrating a cutting angle or depth of a microkeratome accordingly, but this requires additional expensive equipment and special surgical skills. Another problem is that the thickness of the stroma varies from the centre of the cornea towards the edges, and this means that a cutting depth determined for one part of the cornea may not be appropriate at other parts of the cornea. This problem can be addressed by using expensive computer-controlled cutting means together with continuous cutting depth measurement and variation of cutting depth.

### BRIEF SUMMARY OF THE DISCLOSURE

The present applicant has noted that, in humans, the distance from the posterior surface of the corneal endothelium to Descemet's membrane is surprisingly constant. This is irrespective of the subject's age and size and the time of day, and also across a large area of the cornea. Accordingly, a surprising and highly advantageous improvement in cutting accuracy when preparing an endothelial graft is obtained by using the posterior surface of the endothelium as a datum point for determining cutting height (rather than cutting depth, as in the prior art). This is in complete contrast to known techniques, which use the anterior surface of the epithelium as the datum point. By setting a cutting height as measured from a datum point defined as the posterior surface of the endothelium, it is almost guaranteed that a single cutting step will produce a graft comprising essentially the endothelium and Descemet's membrane without unwanted anterior layers of stroma. This can all be achieved without any special adjustments or calibrations by the operator, and no expensive optical measuring apparatus is required. In effect, all that is required is a specially precalibrated and preset combination of a microkeratome and an artificial anterior chamber.

According to a first aspect of the present invention, there is provided a method of separating a posterior endothelial layer of a donor cornea having an epithelium, layers of stroma, a Descemet's membrane and an endothelium, after the donor cornea has been harvested from a cadaver, the method comprising the steps of:

mounting the donor cornea on an artificial anterior chamber provided with a datum point and means for receiving a microkeratome;
using the microkeratome to separate the posterior endothelial layer of the donor cornea;
wherein the artificial anterior chamber is configured such that the datum point is located at a predetermined height relative to the endothelium when the donor cornea is mounted;
and wherein a cutting height of the microkeratome is set to a predetermined height with reference to the datum point.

The posterior endothelial layer may comprise just the endothelium, but in most cases will comprise the endothelium and Descemet's membrane, possibly with a very thin layer of stromal cells.

The datum point may be at the same level as the endothelium, or may be a predetermined distance above or below the endothelium when the donor cornea is mounted on the artificial anterior chamber. What is important is that the datum point provides a physical point relative to which the plane of the endothelium can be determined without requiring the use of special measuring and calibration equipment, and relative to which datum point a cutting height of a microkeratome can be set so as to allow easy separation of the posterior endothelial layer from the rest of the donor cornea.

The datum point may be a surface, for example an annular surface upon which the posterior surface of the donor cornea rests when the donor cornea is mounted on the artificial anterior chamber. Alternatively or in addition, the datum point may be a point or surface having a fixed, predetermined resolved distance from at least a portion of the posterior surface of the donor cornea when this is mounted on the chamber.

According to a second aspect of the present invention, there is provided an artificial anterior chamber device including an annular surface for mounting a donor cornea having an epithelium, layers of stroma, a Descemet's membrane and an endothelium, the annular surface for supporting an annular portion of the endothelium, wherein the annular surface defines a predetermined datum, wherein the device further comprises means for receiving a microkeratome, and wherein the means for receiving the microkeratome is spaced from the datum defined by the annular surface by a predetermined amount such that a cutting height of the microkeratome is determined relative to the datum and not relative to the epithelium.

According to a third aspect of the present invention, there is provided a microkeratome for use with an artificial anterior chamber for cutting at least an endothelium, and preferably an endothelium and Descemet's membrane, from a donor cornea, the microkeratome comprising a housing, means for moveably retaining a cutting part in or on the housing, and means for attaching the microkeratome to the artificial anterior chamber, adapted such that a cutting height of the cutting part is defined relative to a fixed datum on the artificial anterior chamber.

The housing may comprise a casing provided with means for slidably retaining the cutting part in the casing.

Alternatively, the cutting part may be rotatably or pivotally mounted on the housing, for example on a pin or the like, with the cutting part being configured so as to be able to swing through a cutting arc at a predetermined cutting height.

The device may comprise means for adjusting the spacing of the microkeratome, or at least a blade thereof, relative to the datum. The means preferably includes display means to allow an operator to select and set a predetermined spacing with ease. For example, the means may be micrometer screw means or a Vernier gauge means or the like. It is important to appreciate that the spacing from the datum is the only measurement that needs to be taken into consideration by the operator. In particular, no measurement of a distance from the epithelium to the endothelium of the donor cornea, or indeed any depth measurement in relation to the donor cornea, is required.

The microkeratome may be operated by hand, or may incorporate a cutting motor or a vibration or ultrasound motor. The cutting motor or vibration or ultrasound motor may be configured to cause a blade of the microkeratome to oscillate, thereby facilitating cutting. However, it will be appreciated that, when specialised ultra-sharp blades are used, such oscillating motion may not be required, and the microkeratome can then be operated by hand without oscillation of the blade.

Preferably, the microkeratome comprises a cutting part slidably mounted in a casing, the casing being securely mountable on the artificial anterior chamber such that the cutting height relative to the datum point is easily set to a predetermined value. The casing may be screwed on to the artificial anterior chamber, or may be bolted on with two or more bolts or the like. Alternatively, a finely-engineered press or friction fit may be employed. The cuffing part of the microkeratome preferably comprises a blade with a cutting edge that is angled in the cutting plane relative to a cutting direction, and a shoe, having an angled tip that is substantially parallel to the angled part of the blade. An underside of the shoe contacts the epithelial surface of the donor cornea when mounted on the artificial anterior chamber and results in a substantially planer epithelial surface being offered to the cutting edge during cutting. In other words, the cutting part of the microkeratome is analogous to a plane used in woodworking. The casing includes a slot adapted snugly and slidably to receive the cutting part. The cutting part may be provided with a thumb or finger piece at one end to allow the cutting part to be pushed through the casing, for example through the slot, in a smooth and controlled manner. Alternatively or in addition, the cutting part may include a cutting motor to oscillate the blade, or a vibration or ultrasound generator to vibrate the blade. In these embodiments, the cutting part is provided with a handle or some other means to allow it to be moved in the slot. The casing may additionally include means for retaining the cutting part within the slot after use, for example a spring-loaded pin that engages with a notch formed on one side of the cutting part, thereby to prevent re-use or accidental injury to an operator.

The casing may comprise two parts: a lower part which fits onto the artificial anterior chamber and which defines a cutting plane or datum, and an upper part which is secured to the lower part and which, together with the lower part, defines the slot in which the cutting part is slidably received.

Alternatively, the cutting part may be rotatably or pivotally mounted on the housing, for example on a pin or the like, with the cutting part being configured so as to be able to swing through a cutting arc at a predetermined cutting height. The housing is preferably adapted to be securely mountable on the artificial anterior chamber, broadly as described above, such that the cutting height relative to the datum point is easily set to a predetermined value. The cutting part comprises a blade as hereinbefore described, with an optional shoe. A motor or vibrator or ultrasonic oscillator may be provided as before so as to assist with cutting, for example by oscillating the blade.

For a better understanding of the present invention and to show how it may be carried into effect, reference shall now be made by way of example to the accompanying drawings, in which:

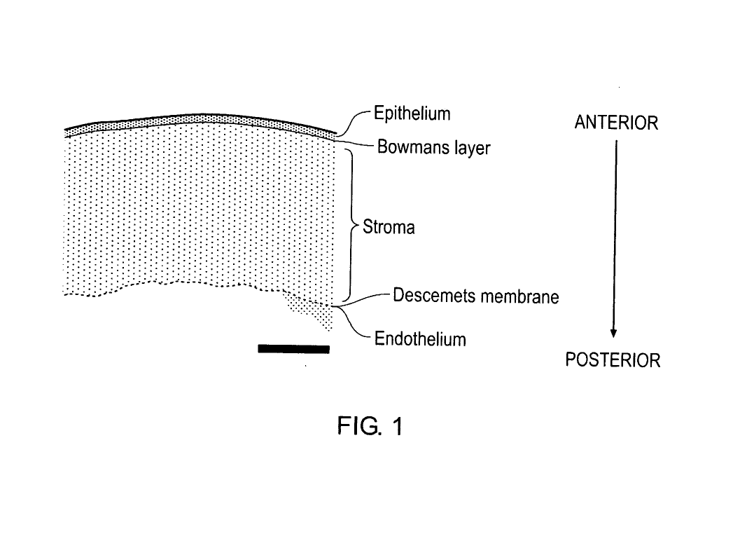

FIG. 1 is a cross-section through a typical human cornea;

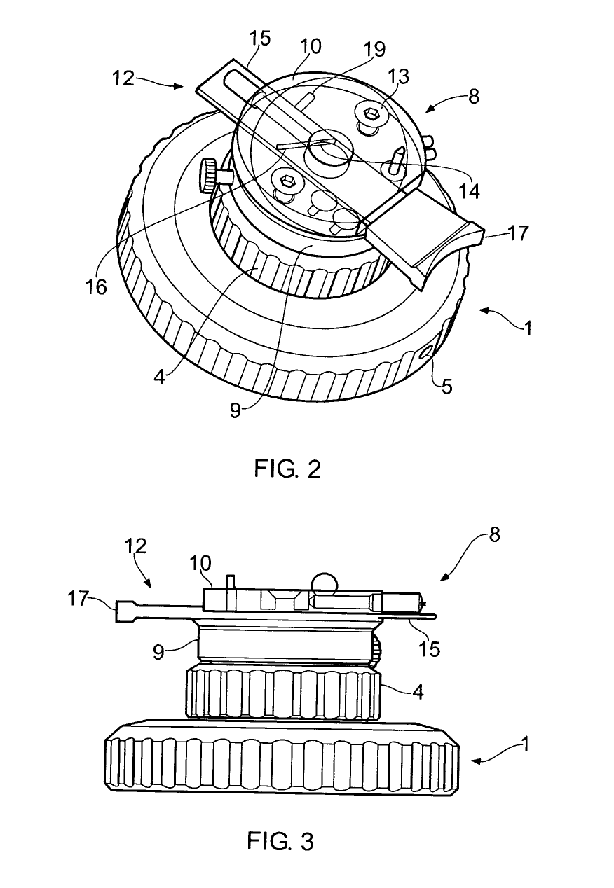

FIG. 2 shows a perspective view of a currently preferred embodiment of the invention;

FIG. 3 shows a side profile of the embodiment of FIG. 2;

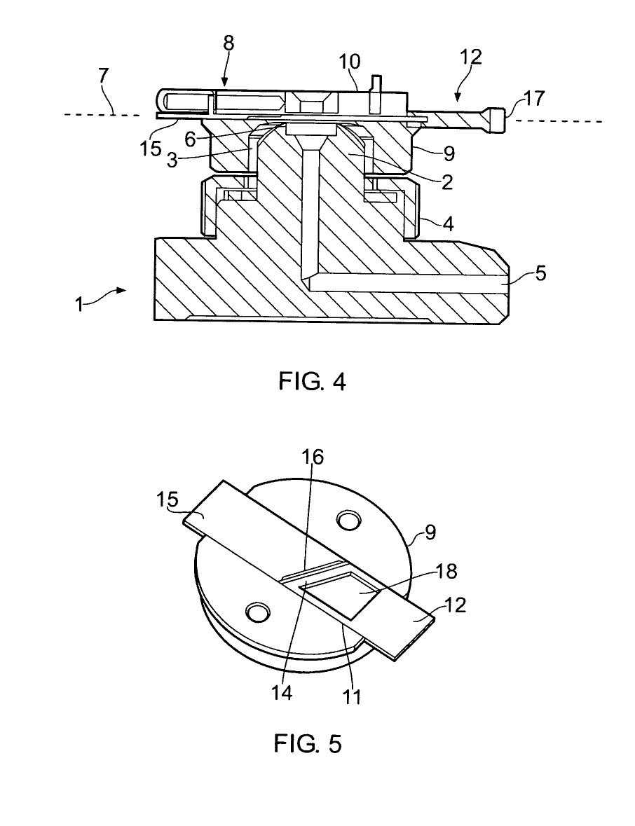

FIG. 4 shows a vertical section through the embodiments of FIGS. 2 and 3;

FIG. 5 shows microkeratome of the embodiments of FIGS. 2 to 4;

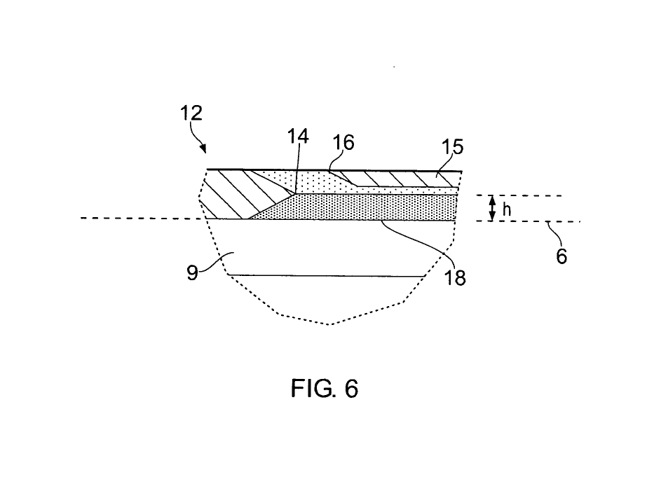

FIG. 6 shows, in cross-section, a blade of the microkeratome of FIG. 5;

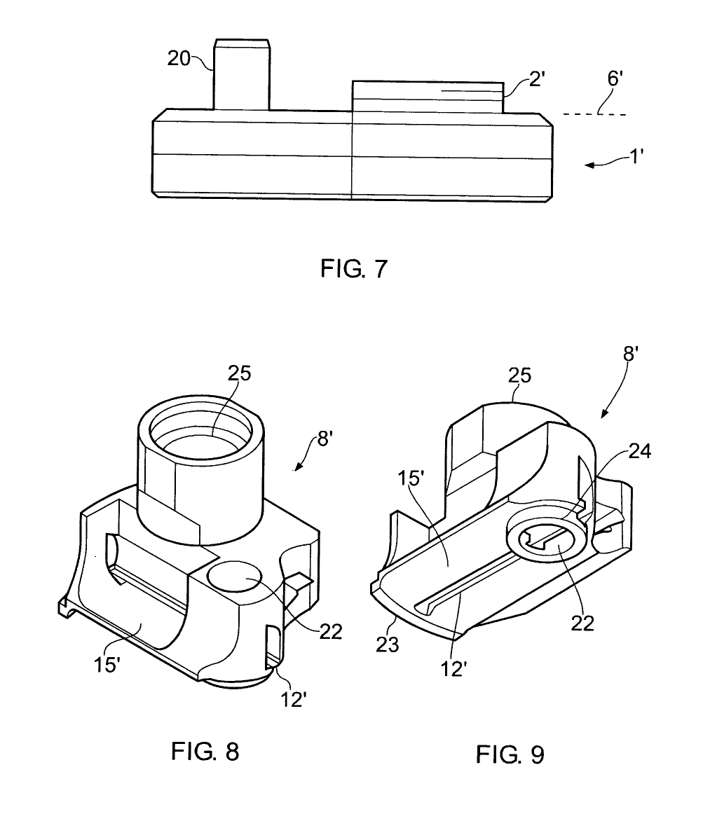

FIG. 7 shows an artificial anterior chamber of a further embodiment of the invention provided with a pivot; and

FIGS. 8 and 9 show a microkeratome for use with the artificial anterior chamber of FIG. 7.

FIG. 1 is a cross-section through a human cornea, showing (from anterior to posterior), the epithelium, Bowman's layer, the stroma, Descemet's membrane, and the endothelium. The total structure is approximately 550 microns thick at the centre of the cornea. Embodiments of the present invention are directed towards methods and devices for cutting the endothelium and Descemet's membrane from the anterior layers of the cornea.

FIGS. 2, 3 and 4 show a currently preferred embodiment of the present invention in various views. There is shown an artificial anterior chamber 1 with a pedestal 2 upon which a donor cornea (not shown) may be mounted, a cap 3 which fits over the pedestal 2 and holds the donor cornea in place, and a locking ring 4 which secures the cap 3 to the chamber 1. Means 5 for supplying fluid under pressure to the chamber 1 is also provided, the pressure being adjustable so as to ensure that the donor cornea is well-supported. A datum plane 6 is defined as the plane containing an annular surface 7 upon which an endothelial surface of the donor cornea rests when mounted on the pedestal 2.

With reference now also to FIGS. 5 and 6, a microkeratome 8 comprising a casing having lower 9 and upper 10 parts is adapted to be fitted snugly, or otherwise secured, to the pedestal 2 over the cap 3. The lower part 9 includes a rebated section 11 that forms a guide or slot in which a blade 12 of the microkeratome is snugly and slidably received. The upper part 10 of the casing is secured to the lower part by bolts 13 and secures the blade 12 within the casing. The blade includes an angled cutting edge 14 and a shoe 15 with a correspondingly angled tip 16. A thumb or finger piece 17 is provided at one end of the blade 12 so as to allow the blade 12 to be pushed through the casing. An edge of the blade 12 within the casing is provided with a notch or rebate into which a spring-loaded retaining pin 19 engages when the blade 12 has been pushed through the microkeratome 8.

FIG. 6 shows the cutting edge 14, the shoe tip 16, and a surface 18 of the rebated section 11 along which the blade 12 slides when operated by pushing the thumb or finger piece 17. The surface 18, which is in the same plane as the annular surface 7 of the chamber 1 when the microkeratome 8 is correctly mounted, thus acts as a guide to the datum plane 6. As can be seen, the cutting edge 14 is a fixed and predetermined height h above the datum plane 6, and by being carefully engineered during manufacture, allows just the endothelium and Descemet's membrane to be cut from the posterior side of the donor cornea without requiring user or operator measurement or other difficult calibrations.

FIG. 7 shows an alternative artificial anterior chamber 1′ in schematic form, including a pedestal 2′ and a datum plane 6′. The artificial anterior chamber 1′ is in most respects similar to that of FIG. 2, but additionally includes a vertically oriented pin 20 upon which a pivoting microkeratome may be mounted.

FIGS. 8 and 9 show a pivoting microkeratome 8′ adapted for use with the artificial anterior chamber 1′ of FIG. 7. The microkeratome 8′ includes an internal cylindrical sleeve 22 which is adapted to fit snugly yet rotatably on the pin 20 of the artificial anterior chamber 1′. The microkeratome also includes a blade 12′ and a shoe 15′, and spacer means 23 and 24 which determine a height of a cutting edge of the blade 12′ above the datum plane 6′. When the microkeratome 8′ is correctly mounted on the pin 20 of the artificial anterior chamber 1′, with the spacer means 23, 24 contacting the datum plane 6′, the microkeratome 12′ may be swung through an arc about the pin 20 so that the blade 12′ describes a cutting arc at a predetermined height above the datum plane 6′.

The microkeratome 12′ additionally includes a receptacle 25 adapted to receive a cutting motor or vibration or ultrasound generator (not shown) for oscillating the blade 12′.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A method of separating a posterior endothelial layer of a donor cornea having an epithelium, layers of stroma, a Descemet's membrane and an endothelium, after the donor cornea has been harvested from a cadaver, the method comprising the steps of:
mounting the donor cornea on an artificial anterior chamber provided with a datum point and means for receiving a microkeratome;
using the microkeratome to separate the posterior endothelial layer of the donor cornea;
wherein the artificial anterior chamber is configured such that the datum point is located at a predetermined height relative to the endothelium when the donor cornea is mounted;
and wherein a cutting height of the microkeratome is set to a predetermined height with reference to the datum point.

2. A method according to claim 1, wherein the datum point is at the same level as the endothelium when the donor cornea is mounted on the artificial anterior chamber.

3. A method according to claim 1, wherein the datum point is at a predetermined distance above or below the endothelium when the donor cornea is mounted on the artificial anterior chamber.

4. A method according to claim 1, wherein the datum point defines a datum plane.

5. A method according to claim 1, wherein the datum point is an annular surface upon which the posterior or endothelial surface of the donor cornea rests when the donor cornea is mounted on the artificial anterior chamber.

6. A method according to claim 1, wherein the datum point is a point or surface having a fixed, predetermined resolved distance from at least a portion of the posterior or endothelial surface of the donor cornea when this is mounted on the chamber.

7. An artificial anterior chamber device including an annular surface for mounting a donor cornea having an epithelium, layers of stroma, a Descemet's membrane and an endothelium, the annular surface for supporting an annular portion of the endothelium, wherein the annular surface defines a predetermined datum, wherein the device further comprises means for receiving a microkeratome, and wherein the means for receiving the microkeratome is spaced from the datum defined by the annular surface by a predetermined amount such that a cutting height of the microkeratome is determined relative to the datum and not relative to the epithelium.

8. A device as claimed in claim 7, comprising means for adjusting the spacing of the microkeratome, or at least a blade thereof, relative to the datum.

9. A device as claimed in claim 8, wherein the means for adjusting includes display means to allow an operator to select and set a predetermined spacing.

10. A device as claimed in claim 7, wherein the spacing of the microkeratome, or at least a blade thereof, is preset relative to the datum and is not adjustable by an operator.

11. A microkeratome for use with an artificial anterior chamber for cutting at least an endothelium, and preferably an endothelium and Descemet's membrane, from a donor cornea, the microkeratome comprising a housing, means for moveably retaining a cutting part in or on the housing, and means for attaching the microkeratome to the artificial anterior chamber, adapted such that a cutting height of the cutting part is defined relative to a fixed datum on the artificial anterior chamber.

12. A microkeratome as claimed in claim 11, comprising means for adjusting the cutting height relative to the datum when the microkeratome is mounted on the artificial anterior chamber.

13. A microkeratome as claimed in claim 11, wherein the cutting height is preset relative to the datum and is not adjustable by an operator when the microkeratome is mounted on the artificial anterior chamber.

14. A microkeratome as claimed in claim 11, wherein the cutting part of the microkeratome comprises a blade with a cutting edge that is angled in a cutting plane relative to a cutting direction.

15. A microkeratome as claimed in claim 11, wherein the cutting part of the microkeratome comprises a blade with a cutting edge that is perpendicular in a cutting plane relative to a cutting direction.

16. A microkeratome as claimed in claim 11, wherein the cutting part further comprises a shoe, the shoe having an underside that contacts the epithelium of the donor cornea when mounted on the artificial anterior chamber

17. A microkeratome as claimed in claim 16, wherein the shoe has a tip that is substantially parallel to the angled cutting edge.

18. A microkeratome as claimed in claim 11, wherein the housing includes a slot adapted snugly and slidably to receive the cutting part.

19. A microkeratome as claimed in claim 18, wherein the casing comprises a lower part which fits onto the artificial anterior chamber and which defines a cutting plane or datum, and an upper part which is secured to the lower part and which, together with the lower part, defines the slot in which the cutting part is slidably received.

20. A microkeratome as claimed in claim 11, wherein the cutting part is provided with means for moving the cutting part relative to the housing.

21. A microkeratome as claimed in claim 11, wherein the housing additionally comprises means for retaining the cutting part within the housing after use.

22. A microkeratome as claimed in claim 21, wherein the means for retaining comprises a spring-loaded pin that engages with a notch formed on one side of the cutting part.

23. A microkeratome as claimed in claim 11, wherein the cutting part is pivotally mounted on the housing.

24. A microkeratome as claimed in claim 11, wherein the cutting part is slidably mounted in the housing.

25. A microkeratome as claimed in claim 11, wherein the microkeratome includes at least one item from the group comprising: vibration generating means, ultrasound generating means, and a cutting motor.
